(19) United States
(12) Patent Application Publication
(10) Pub. No.: US 2004/0157211 A1
(43) Pub. Date: Aug. 12, 2004

(54) Method and a system for counting cells from a plurality of species

(76) Inventors: Ole Skyggebjerg, Hillerod (DK); Martin Glensbjerg, Bronshoj (DK)

Correspondence Address: Ronald W Citkowski, Gifford Krass Groh Sprinkle Anderson & Citkowski, Suite 400, 280 North Old Woodward Avenue, Birmingham, MI, 48009, US

(21) Appl. No.: 10/480,968

(22) PCT Filed: Jun. 10, 2002
(86) PCT No.: PCT/DK02/00389

(30) Foreign Application Priority Data
Jun. 8, 2001 (DK) PA 2001 00896

(51) Int. Cl. C12Q 1/70; C12Q 1/04; G01N 1/30; G01N 33/48

(52) U.S. Cl. 435/5; 435/34; 435/40.5

(57) ABSTRACT

The present invention relates to a method and a system for the assessment of cells in a liquid sample. The present method provides individual counts of substantially all the cells in the sample which are susceptible to the method of making cells distinguishable, in a manner whereby it is not necessary to conduct calibrations between assessments of samples with cells from different species within one taxonomic group. This is provided by measuring a part of the cells having substantially identical spatially confined identifiable substances, such as DNA. This makes the method less sensitive to variations in cell size and morphology than prior art methods for cell counting, such as Coulter counting.

The present invention relates to a method and a system for the assessment cells in a liquid sample.

For the purpose of US-national stage, this application is a non-provisional claiming priority from Danish patent application no. PA 2001 00896 filed 8 Jun. 2001, which is hereby incorporated by reference in its entirety. All patent and non-patent references cited in that application, or in the present application, are also hereby incorporated by reference in their entirety.

### BACKGROUND

Detection of cells in a liquid sample is today conducted by several methods, such as manual and automated microscopic methods, methods based on the Coulter technique and flow cytometry.

The manual microscopic methods generally involve staining of cells followed by inspection of one or more view areas where individual cells are counted. The results obtained by these methods are dependent on the training of the operator with respect to identifying individual cells.

When the microscopic method includes an automatic image processing tools the operator dependency is removed. On the other hand, the ability of the image processing system to identify and count cells becomes important. When dealing with plurality of cell species this generally requires extensive calibration of the system. Another aspect of the microscopic methods is that the total volume of sample which is analysed is generally low, typical volume would be about 0.1 μl. This affects the statistical property of the result.

Methods based on the Coulter counter principle detect individual cells as variation in electrical conductivity while the sample flows through a narrow channel.

In flow cytometry a predetermined volume of the liquid is arranged to flow so that substantially only one cell is passing a detector at a time. The flow cytometry system has to be calibrated each time the type of the sample changes, i.e. if for example the cells in the sample differs from the cells in the former sample. Flow cytometry requires expensive systems, and furthermore as explained above shifting of samples requires a labour-consuming calibration before reliable results can be obtained.

In common for all these systems is that cells generally have to be intact physically or morphologically during analysis. Since many cells, such as an example mammalian cells and many bacteria, have tendency to form colonies various methods are used to break up these colonies prior to analysis. These methods are generally time consuming since the conditions used have to preserve cell membranes while breaking up the bindings between cells.

### SUMMARY OF THE INVENTION

The present invention relates to a method for the assessment of the number of cells in a liquid sample, said sample comprising at least one species of cells being selected from a variety of species, cells of each of said species being substantially identical with respect to morphological properties while the inter-species morphological properties may vary, each cell being assessed containing substantially identical spatially confined identifiable substances, such as DNA, comprising the steps of

establishing conditions for making the identifiable substances susceptible to being distinguishable,

establishing conditions providing substantially spatial separation of at least the spatially confined identifiable substances being susceptible for distinguishing,

optionally staining at least substantially all spatially confined identifiable substances,

identifying information relating to substantially each individual cell to be assessed, and

correlating the information to the number of individual cells in the samples.

The present method provides individual counts of substantially all the cells in the sample which are susceptible to the method of making cells distinguishable, in a manner whereby it is not necessary to conduct calibrations between assessments of samples, preferably whereby it is not necessary to conduct calibration between assessments of different samples comprising cells from different species within one taxonomic group. This is provided by measuring a part of the cells having substantially identical spatially confined identifiable substances, such as DNA. This makes the method less sensitive to variations in cell size and morphology than prior art methods for cell counting, such as Coulter counting.

Therefore, it is normally not necessary to calibrate the system when a sample from e.g. another species within the same taxonomic group, or another cell-line within the same species is assessed. As the variation in the spatially confined identifiable substances among different species within one taxonomic group is substantially low it is also not necessary to calibrate the system when a sample from another species within the same taxonomic group is assessed. As an example it is possible according to the method of the present invention to measure a sample of mammalian cells regardless of its species by using a method which is adapted to give a signal in accordance with the amount of DNA present in the cell. The variation in the amount of DNA and structure of the cell nuclei in various species of mammalian cells does not require a calibration according to which species is being measured when analysed by a method according to the present invention.

Thereby the present invention allows that measurements of samples of different species may be conducted sequentially without any calibration of the system, such as a method of assessing the number of cells in a sample comprising at least a first species of cells being selected from a variety of species within a given taxonomic group by the method described above, and subsequently without any calibration or training performing the method with a sample comprising a second species of cells being selected from a variety of species, wherein the first species of cells and the second species of cells are different with respect to morphological properties, and preferably belong to the same taxonomic group.

According to a further aspect, the invention relates to a system for the assessment of the number of cells in a liquid sample, said sample comprising at least one species of cells being selected from a variety of species, cells of each of said species being substantially identical with respect to morphological properties, while the inter-species morphological properties may vary, each cell being assessed containing substantially identical spatially confined identifiable substances, comprising:

a device comprising at least

sample receiving means for receiving said sample

a first preparation chamber

sample flow means for delivering said sample to said first preparation chamber,

at least a first one reagent receiving means for receiving a first reagent,

a first reagent flow means for delivering first reagent from said first reagent

receiving means to said first preparation

chamber,

a second reagent receiving means for receiving a second reagent,

a second reagent flow means for delivering second reagent from said second reagent receiving means,

a compartment comprising an exposing domain,

a flow channel for delivering said sample and said reagent to said compartment,

a detection device for detecting information relating to each spatially confined identifiable substances,

processing means for processing the information detected,

presentation means for presenting the processed information as a number of cells in the sample.

The system can be used for the assessment of cells according to the invention.

### DRAWINGS

FIG. 1 shows the result of a comparison test between a manual total cell count and the cell count with a method according to the invention using NSO mouse myeloma cell samples from a 10-Liter bioreactor. The correlation coefficient is 0.96, indicating that there is a good correlation between the two methods.

FIG. 2 shows the viability data from the same experiments. The cell samples were analysed with a method according to the invention before and after treatment with a lysis buffer, giving an estimate of dead and total cells, respectively. The manual counting method was based on the Trypan blue exclusion procedure.

FIG. 3 is a comparison plot of triplicate data from a CHO HIR cell line, showing that with a method according to the invention varies much less than those of the manual microscopy method. The CV of the NucleoCounter is less than 5%.

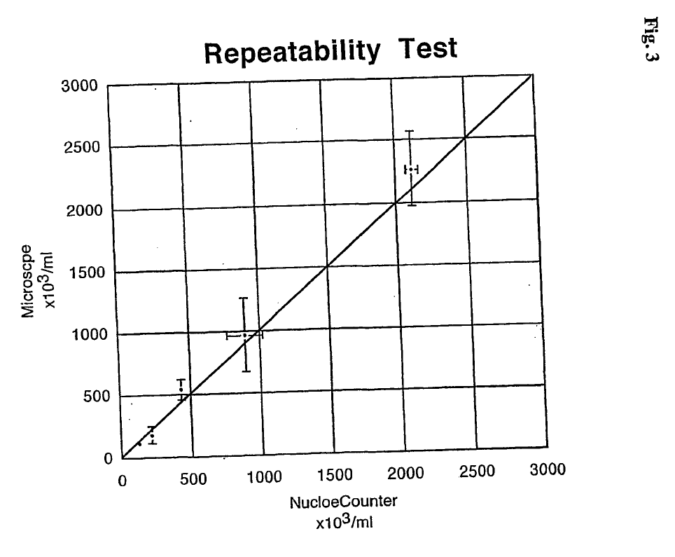

FIG. 4 is a comparison of a cell count of two samples exposed to ultrasound treatment and the effect of such treatment on the cell count as a function of the duration of the treatment.

FIG. 5 shows an example of a print out of one exposure from a sample to an array of detection elements. Each white spot corresponds to one nucleus in the sample compartment. The section which is blown-up in the figure illustrates approximately the field of view in traditional microscopy methods. The effect of low magnification is clearly visible in the limited spatial representation of each cell, which only corresponds to a few pixels.

### DETAILED DESCRIPTION

Samples

The method according to the invention may be used for assessing a variety of species of cells in a variety of liquids, such as liquids, wherein the variety of species of cells is selected from one of the following taxonomic groups: animal cells, yeast cells, fungal cells, plant cells, algae, plasmodia, bacteria, virus. More preferably, the variety of species are animal species including mammalian species, fish, insect and reptiles. More preferably, the variety of species of cells are selected from mammalian species. The sample as such may be selected from cultures of cells grown in cultures media, waste water, body fluids such as blood or urine.

Culture media is often assessed with respect to total cell count, as well as count of dead and living cells, to obtain information about for example a fermentation process. The sample can be obtained from a fermentation reactor, or from culturing flasks, or T-bottles and the like, or directly from body-fluids and waste water, bathing water etc.

The variety of cells mentioned above provides a wide variety of morphological differences of the cells, such as differences in size, shape, symmetry. By the term “inter-species morphological difference” is meant the difference between cells of one species to the cells of another species within one taxonomic group, such as the difference between two types of mammalian cells, difference between two types of yeast cells or two types of bacterial cells or two types of plant cells.

Generally, the variation between cell species within the different taxonomic groups, the species being mammalian, bacterial, plant, yeast, or bacteria or the like, are such that calibration or adjustment based on apriori or observed information is not necessary. Embodiments of the present invention allows the counting of a plurality of different species within a given taxonomic group.

Mammalian cells can differ with respect to many physical and/or morphological properties. Many methods of the prior art for the counting of mammalian cells are therefore dependent on a calibration or adjustment in order to count reliably the number of cells present in a sample.

According to an especially preferred embodiment, the invention relates to the assessment of the number of cells in a sample of mammalian cells. One common property of mammalian cells containing nuclei is the presence of an approximately stable amount of DNA. Since the principle according to one embodiment of the present invention for the counting of mammalian cells is based on staining the DNA within the nuclei, the method is reliable in the measurement of various cell species without any adjustment or calibration.

One of the most pronounced inter-species morphological difference is the size of the cells. Many embodiments of the present invention allow a reliable assessment of cells under conditions where the inter-species morphological difference is the variation in size on the order of factor 1.2, preferably more than a factor 1.2 such as a factor 1.5, more preferably a factor of 2 or even a factor 4, or more than 4 such as 6 or 8 or more or even as much as a factor of 10 or more. Within these ranges the method according to the present invention finds its application without any additional adjustment or calibration. Samples being analysed can either contain cells of different species to be analysed in one measurement or the analysis of two or more different samples containing cells from different species can be analysed under identical conditions.

In another embodiment the inter-species morphological difference may be a variation in shape selected from rod-like, spherical, circular, star-shaped with spikes. Such morphological difference often also result in difference in symmetry between two species of cells.

Within one species of cells, the cells are substantially identical with respect to at least the morphological properties as discussed above. The sample comprises at least one species of cells, however the sample may also comprise at least two different species of cells, such as at least three different species of cells, such as at least four different species of cells, so that the sample may comprise cells exhibiting a wide variation with respect to the morphological properties discussed above.

Spatially Confined Identifiable Substance

Although the cells vary as described above, they preferably all have a spatially confined identifiable feature or substance, that is capable of being identified, optionally by staining. The spatially confined identifiable substance is preferably a cell nucleus. However, spatially confined identifiable substance may be nucleotides in general independent of their presence in the cells, but it is preferred that the nucleotides are located in the cell nucleus.

Other spatially confined identifiable substances can similarly also be other chemical components of the cells, such as proteins, enzymes, proteoglycanes, sugars, ATP, NADH.

The term spatially confined identifiable substance can thus be interpreted as any chemical, biological or physical feature of the cell which allows for the identification of substantially individual cells. The spatially confined identifiable substance relating to one taxonomic group of species of cells allows the identification of one cell only.

Process

The process comprises at least the following steps:

a) establishing conditions for making the identifiable substances susceptible to being distinguishable,

b) establishing conditions providing substantially spatial separation of at least the spatially confined identifiable substances being susceptible for distinguishing,

c) optionally staining at least substantially all spatially confined identifiable substances,

identifying information relating to substantially each individual cell to be assessed.

By the term “establishing conditions for making the identifiable substances susceptible to being distinguishable” is generally meant to include any pre-treatment of the cells, to be able to distinguish the identifiable substances. Thus, step a) includes for example that if necessary the cells or cell membranes are made permeable to be able to stain nucleotides in the cell. In case of dead and/or dying (non-viable) cells, the cells may be sufficiently permeable to stain nucleus material, whereby step a) merely includes the condition, that if dead cells are to be assessed, no pretreatment may be necessary.

Further embodiments can include any inherent property of the cell, such as active or passive transport of a component into the cells. Such embodiments are generally of interest where a specific identification of cells on the bases of its activity are of interest.

In another embodiment the process of making the cells permeable may include lysing of the cells at conditions at which the cell nucleus is substantially not lysed, whereby the nucleus, or any DNA material within the nucleus may be stained.

In yet another embodiment the condition for making the identifiable substances susceptible to being distinguishable includes selective staining of one or several receptors on the surface of the cells or in the interior of the cells.

By the term “establishing conditions providing substantially spatial separation of at least the spatially confined identifiable substances being susceptible for distinguishing” is meant that the cells or at least the spatially confined identifiable substances of the cells may be arranged so that substantially each cell may be identified as an individual cell. For many of the applications for which this invention is suitable, aggregates of cells are seen in the sample. These aggregates can be separated by means known to the person skilled in the art, such as by adding a substance to the sample such as an enzyme, wherein said substance is capable of separating the aggregates. These methods are generally time consuming and it-is therefore often preferred to substantially remove the identifiable substances from the cells prior to analysis for instance by selective lysing of cell membranes. In one embodiments the aggregates are broken apart by lysing the cells, at conditions at which the cell nucleus is not lysed. When step b) Is conducted by lysing, step a) and b) may be conducted as one step. In the following the term “prior to analysis” is used synonymously with the term “prior to identifying information relating to substantially each individual cell to be assessed”.

Thus, in case of lysing the lysing conditions are preferably selected to obtain lysing of cell membranes, but not lysing of nucleus of the cell. One preferred method which performs the lysing in only few seconds is one where a reagent is added to the sample to obtain a concentration of Triton X-100 of around 1%, preferably in the range between 0.1% and 2%, and a concentration of citric acid of around 0.15 to 0.25 mM, preferably in the range between 0.1 mM and 1 mM. The pH of the final solution is preferably low, often between 2 and 6, preferably substantially determined by the pH which results in the addition of citric acid on its acid form. Often when the sample has been treated as described above it is ready to be analysed within only few seconds after the mixing of the solution. Preferred embodiments further require the adjustment of the pH prior to analysis, often due to a pH dependence of a staining component. An example is when using Propidium Iodide as DNA staining dye its fluorescence property is dependent on pH, generally requiring pH above 6.

In several embodiments of the present invention, the chemical component Triton X-100 (t-Octylphenoxy polyethoxyethanol) is added to the sample prior to analysis. Triton X-100 is a detergent, and its ability to lyse cell membrane has been used for a long time. It has also been found that Triton X-100 has positive effect on dissolving cell aggregates. In the present invention it is preferred that Triton X-100 is present in quantities in the final solution of between 0.05% and 5%, preferably between 0.1% and 2%, more preferably between 0.2% and 1.5%, more preferably between 0.75 and 1.25%, such as approximately 1%.

Another chemical component which is preferred in the present invention is CPC (Cetyl Pyridinium Chloride). CPC has been found to be an effective lysing agent as well as having positive effect on the dissolution of cell aggregates. Many preferred embodiments include CPC in concentration between 0.05% and 5%, more preferably between 0.1% and 4%, such as 0.1% to 3%, such as from 0.1% to 2%, more preferably between 0.2% and 1%, such as from 0.5 to 0.75%.

Cells aggregates are often formed by chemically binding two or more cells together, normally in a process which involves one or more proteins. Therefore the use of an enzyme capable of protease activity is generally preferred. In the present invention many embodiments include the addition of such enzyme to the sample prior to analysis. An example of a suitable enzyme is the Trypsin enzyme. Preferred concentrations of Trypsin are between 0.05% and 5%, more preferably between 0.1% and 2%, more preferably between 0.2% and 1%.

In other cases cells stick together to form cell aggregates because of their cell walls.

Such cell walls consist of polymers such as cellulose, hemicellulose, glucans and proteins. Adding a cell-wall degrading reagent will aid in separating these cells from one-another. Preferably said cell-wall degrading reagent comprises at least one enzyme, such as lysozyme, cellulase, pectinase, hemicellulase where the enzyme concentration of the final solution is between 0.05% and 5%, more preferably where the enzyme concentration of the final solution is between 0.1% and 2%, more preferably where the enzyme concentration of the final solution is between 0.2% and 1%. An alternative for these cells is to use a strong acid capable of hydrolysing the cell wall optionally at elevated temperature.

One group of chemical components, from which Pluronic F-127 is a typical example, are often added to biological samples in order to prevent aggregation. These chemical components can saturate the sites on the surface of the cells which normally are an important factor in forming of cell aggregates.

Citric acid is a small organic molecule with several interesting properties relevant to the present invention. Citric acid is a trivalent acid and as such is therefore commonly used for the adjustment of pH in a buffer solution. Another property of interest is the complex binding efficiency of citrate. Many preferred embodiments include citric acid, preferably where the Citric acid concentration of the final solution is between 1 mM and 1000 mM, such as between 1 mM and 500 mM, more preferably between 50 and 250 mM, such as between 100 and 200 mM, such as 125 mM to 175 mM, for example approximately 150 mM. In other cases a lower amount of Citric acid suffices for example between 10 mM and 100 mM, more preferably between 20 mM and 50 mM.

Many chemical components have the property of complex binding. In addition to citric acid another and often preferred chemical component is EDTA.

For many of the embodiments of the present invention the pH of the sample is of interest. The pH of a solution can often be determined by the composition of the sample but in many preferred embodiments of the present invention the pH of a sample is adjusted, preferably by the use of a buffer solution which is added to the sample. Such buffers are preferably one or several of the following: Citrate buffer, Phosphate buffer, HEPES buffer.

When the pH of a sample is adjusted the pH which is sought can vary. Often the pH is around neutral pH of about 7 but in may preferred embodiments the pH can be between 1 and 7, preferably between 2 and 6, more preferably between 3 and 5. In jet other embodiments a higher pH is sought, such as is between 3 and 9, preferably between 4 and 8, more preferably between 5 and 7.

Step e) is made optional, since for some applications and/or methods, the cells may be identified without staining. However, if necessary the staining is conducted so that at least the spatially confined identifiable substances are stained of substantially all the cells present in the sample.

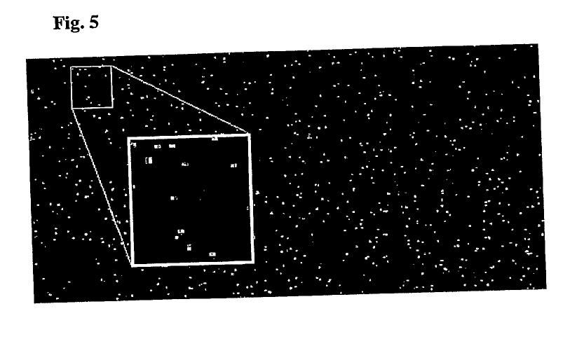

As discussed above some of the cells of interest show properties which can be used to form and detect a spatially resolved electromagnetic radiation without substantially any addition of any reaction component. The term “detection of spatially resolved electromagnetic radiation” corresponds to what may be generally regarded as forming an image on an array of detection elements, although in a strict sense no image is formed on the array of detection elements. However from the signals detected by the array an image may be printed using state of the art methods. One example of such an “image” is shown in FIG. 5.

The signals which often can be formed and detected in this manner are signals which are substantially caused by attenuation of electromagnetic signal, and/or by emission of electromagnetic irradiation by photoluminescence. The attenuation of signals and/or the photoluminescence signals being associated to one or more molecules which is/are a part of the cell, such as DNA and/or proteins.

Thus, for many of the embodiments of the invention the method includes a step of staining before detection or identification. By staining is meant addition of molecules giving rise to one or several of the following phenomena: attenuation of electromagnetic radiation, photoluminescence when illuminated with electromagnetic radiation, scatter of electromagnetic radiation, raman scatter.

Such staining may be selected from one or more nucleic acid dyes and/or one or more potentiometric membrane dyes is added, such as selected from acridine orange CAS-65-61-2/CAS-10127-02-3), cyanine dyes (e.g. TOTO™-1 iodide CAS#: 143 413-84-7-Molecular Probes, YO-PRO™-1 iodide CAS#: 152 068-09-2-Molecular Probes), indoles and imidazoles (e.g. Hoechst 33258 CAS#: 023 491-45-4, Hoechst 33342 CAS#: 023 491-52-3, DAPI CAS#: 28718-90-3, DIPI (4′,6-(diimidazolin-2-yl)-2-phenylindole in particular propidium iodide CAS#: 25535-16-4.

The preferred amount of any chemical component added can be varied according to the properties of the particles being assessed. Of the amount can be more than 30 μg per ml of the sample, but often it is preferable to have amount of less than 30 μg per ml of the sample, even less than 10 μg per ml of the sample. Some aspects of this invention allow an amount of less than 1 μg, or even less than 0.1 μg per ml of the sample.

Further improvement of the separation of cells and/or spatially confined identifiable substances can be obtained by mechanical treatment of the samples including but limited to vigorous shaking and mixing, cutting, subjection to high/low pressure, and sonication.

In particular it has been observed that subjecting samples containing cell aggregates to ultrasound treatment improves the separation of cells from each other and especially improves the separation of nuclei from one another, thus giving a more accurate count of the number of cells in the sample because more nuclei are separated from the aggregates.

One factor of importance to this method is the effect of the ultrasonic treatment. This effect can be difficult to assess especially in commercially available laboratory equipment since a homogenous ultrasonic effect is very difficult to obtain. One preferred method to determine the effect of such ultrasonic treatment is to subject a sample of virtually untreated mammalian cells to the treatment for a period of between a few seconds and a few minutes and to observe that virtually all cells and even cell nuclei have been disrupted.

When using an ultrasonic water bath one method to assure optimal effect from a given water bath is to adjust the water level of the sonication bath, where it is preferably adjusted so that vigorous ripples are seen on the surface. Preferably, ultrasound treatment is performed for a period from 20 seconds to 5 minutes, more preferably from 30 seconds to 2 minutes.

Preferably the ultrasound treatment is performed after adding a reagent to the cells causing the nucleus not to disintegrate upon ultrasound treatment, preferably wherein said reagent has the effect of adjusting the pH of the sample, preferably where the reagent comprises acid such as citric acid.

Conveniently, the ultrasound treatment provides an effect of 50 to 120% of the effect required to destroy the cells under similar circumstances.

Identification of Information

By the term “identifying information relating to substantially each individual cell to be assessed” is meant that signals relating to substantially each individual cell is identified, wherein the signals may relate to the cell as such or to the parts of the cell.

Embodiments of the present invention make use of the interaction of electromagnetic radiation with the spatially confined identifiable substance.

In one preferred embodiment information relating to each individual cell to be assessed is obtained by performing one or more exposures of spatially resolved electromagnetic signals from the sample onto an array of active detection elements, such as detection of electromagnetic signals. In particular the information relating to cells is fluorescence signals, wherein the fluorescence signals may relate to the spatially confined identifiable substances as such, or to the staining added to the sample.

Sample Compartment

In a preferred embodiment the sample is arranged in a sample compartment before identifying information. The sample compartment has a wall part defining an exposing area, the Wall part allowing electromagnetic signals from the sample in the compartment to pass through the wall to the exterior.

A sample compartment, containing the sample being analysed, arranges preferably as much sample volume as possible in such a way that it can be exposed to the array of detection elements, thus allowing the analysis of a large area of the sample simultaneously. One method for accomplishing this is to define the thickness of sample compartment in a direction which is not parallel to the plane of detection elements, thus increasing the effective volume per area of sample compartment exposed to the detection elements. The optimum thickness often being determined by any effective focus depth of a focusing system.

In such cases the sample compartment limits the dimension of the sample in the direction which is substantially not parallel to the plane of array of detection elements, to a thickness of at least 20 μm or less, preferably to a thickness of more than 20 μm, more preferably to a thickness of more than 40 μm, more preferably to a thickness of more than 60 μm, more preferably to a thickness of more than 80 μm, more preferably to a thickness of more than 100 μm, more preferably to a thickness of more than 140 μm, more preferably to a thickness of more than 180 μm, more preferably to a thickness of more than 250 μm, more preferably to a thickness of more than 500 μm, more preferably to a thickness of more than 1000 μm.

For some applications a tubular sample compartment is used whereby it also is possible to increase the area of sample being analysed simultaneously by increasing the radius of such tubular sample compartment.

The sample compartment may be a disposable sampling device as described in PCT/DK99/00605 which is hereby incorporated by reference.

Volume of the Sample

For the individual applications of the present invention, it is mostly possible to define a lower limit of the number of cells to be relevant to assess and thereby a relevant size of volume to be assessed. This is in particular the case when assessing bacteria in the sample as well as somatic cells in the sample.

For some applications also an upper limit is definable, such as somatic cells in blood. Preferably, the size of the volume of the sample is large enough to allow identification therein of at least two particles with the desired statistical quality.

The present invention offers methods which allow for a highly reliable and statistically signification analysis of only a small volume of sample material compared to many of the presently used methods by assessing the presence of cells in a considerably large fraction of the sample material. More preferably, the size of the volume of the liquid sample is sufficiently large to allow identification therein of at least four of the cells. This will correspond to a repeatability error of approximately 50%. Still more preferably, the size of the volume of the liquid sample is sufficiently large to allow identification therein of at least 10 of the cells. This will correspond to a repeatability error of approximately 33%. Even more preferably, the size of the volume of the liquid sample is sufficiently large to allow identification therein of at least 50 of the cells. This will correspond to a repeatability error of approximately 14%. Evidently, where possible, it is preferred to aim at.conditions where the size of the volume allows identification of even higher numbers. Thus, 10 when the size of the volume of the liquid sample is sufficiently large to allow identification therein of at least 100 of the cells, it will correspond to a repeatability error of approximately 10%, and when the size of the volume of the liquid sample is sufficiently-large to allow identification therein of at least 1000 of the cells, it will correspond to a repeatability error of as low as approximately 3%.

In the present context the term “sample” does not necessarily mean the sample present in the compartment, but rather the sample introduced into a flow system connected to the sample compartment according to the invention. It is of interest to minimise the use of sample material and any chemical component used for the analysis. This can be accomplished by the use of the present invention. Sample volumes as small as 5 ml or less and even as small as 0.02 ml can be used. The volume of the sample needed is highly dependent on the number of cells present in the sample and the predetermined statistical quality parameter sought, whereby typical volumes applied is less than 5 ml of a liquid sample, preferably by using less than 2 ml of a liquid sample, more preferably by using less than 1 ml of a liquid sample, more preferably by using less than 0.5 ml of a liquid sample, more preferably by using less than 0.2 ml of a liquid sample, more preferably by using less than 0.1 ml of a liquid sample, more preferably by using less than 0.05 ml of a liquid sample, more preferably by using less than 0.02 ml of a liquid sample, more preferably by using less than 0.01 ml of a liquid sample, the volume being defined as the total volume of any liquid sample introduced to the sample compartment, or any flow system connected to the sample compartment before or after or during the measurement of the sample.

The method and system according to the present invention allows the assessment of samples of a wide variety of volumes. The volume of the sample from which signals are exposed onto the array is normally in the range between 0.01 μl and 20 μl, such as in the range between 0.01 μl and 10 μl, such as in the range between 0.01 μl and 4 μl, such as in the range between 0.02 μl and 10 μl, preferably in the range between 0.04 μl and 2 μl, such as in the range between 0.05 μl and 2 μl, such as in the range between 0.01 μl and 1.50 μl.

A large volume of the sample may be measured by passing the volume of sample through a particle retaining means, such as a filter, electrical field, magnetic field, gravitational field, such means preferably being included in the device or can be arranged to interact with any sample within the device. The particle retaining means should preferably be able to retain substantially all particles present in a sample, or at least a substantially representative fraction of at least one type of particles present in the sample. When the particles from a large sample are retained, those particles can be re-suspended in a volume which is less than the volume of sample passed through the particle retaining means.

The sample is preferably at stand still during the exposure to obtain stand still conditions for the detection means. In one embodiment of the present invention a signal from the cells being analysed is detected while the cells are still substantially retained by a particle retaining means. In such embodiment the particle retaining means are integrated with, or in close connection to a sample compartment.

Detection—Identification of Information

The detection means may comprise any detectors capable of sensing or detecting the signals emitted from the sample. Methods for identifying or detecting information relating to cells are described in for example PCT/DK98/00175, PCT/DK99/00605 and PCT/DK01/00265.

In a preferred embodiment detection means comprises a detector being an array of detecting devices or detection elements, such as a charge coupled device (CCD) the CCD may be a full frame CCD, frame transfer CCD, interline transfer CCD, line scan CCD, an eg. wavelength intensified CCD array, a focal plane array, a photodiode array or a photodetector array, such as a CMOS. The CMOS is preferably a CMOS image sensor with on-chip integrated signal condition and/or signal processing. Independent of the choice of any of the above detection devices the detection means may further comprise a white/black or colour CCD or CMOS. The size of the detection elements determines to some extend its sensitivity. In some applications it is therefore of interest to have detection elements of size of about 1 μm² or less. In certain situations the size of the detection elements in the array of detection elements is less than 20 μm², preferably less than 10 μm², more preferably less than 5 μm², more preferably less than 2 μm². One way of expressing the ratio at which the image should preferably be formed on the array of detection elements is to consider the imaging of an individual cells or identifiable substance of the sample on the detection elements. It is often preferred that the electromagnetic radiation from the individual cells or identifiable substances are exposed on at the most 100 detection elements, such as at the most 81 detection elements, such as at the most 64 detection elements, such as at the most 49 detection elements, such as at the most 36 detection elements, such as at the most 25 detection elements, in particular on at the most 16 detection elements and more preferred at the most 9 detection elements. It is even more preferred that electromagnetic radiation from individual cells or identifiable substances are exposed on at the most 5 detection elements, or even on at the most 1 detection element. The larger number of elements per particle will provide more information on the individual cells or identifiable substances, while the smaller number of elements per cells or identifiable substances will increase the total count that can be made in one detection exposure and make the method less susceptible to variations in size and morphology between individual cells and between cells from different biological species. One example of low resolution exposure is shown in FIG. 5.

The signal which is detected is substantially caused by one or several of the following: photoluminescence with lifetime of the exited state of less than or equal to 10⁻⁶ seconds, photoluminescence with lifetime of the exited state of garter than 10⁻⁶ seconds, chemiluminescence, rayleigh scatter, raman scatter, attenuation of electromagnetic radiation, absorption of the electromagnetic radiation, scatter of the electromagnetic radiation as discussed above.

In several embodiments of the invention the method may be conducted in a single-sided system or a double-sided system as described in PCT/DK01/100265, which is hereby incorporated by reference.

Correlation and Assessment

As discussed above the method and system according to the invention may be used for a wide variety of samples comprising a wide variety of cells from within one taxonomic group without calibration and training. A further advantage is that the method and system according to the invention, may be used without entering any apriori information of the sample and/or expected cells before the correlation and assessment of the number of cells. By apriori information is meant any information about the sample and/or the species of cells that should otherwise be used for the system to correlate the information identified to the number of cells. Also the correlation to the number of cells may be conducted without use of any morphological information about the species of cells derivable from the detection as such, that is morphological information that may be obtainable in addition to the information expected to be identified.

In several preferred embodiments the signals are collected or analysed under conditions which allow the identification of individual cells. Preferably methods of image processing are used which reflect information from an individual cell into only one detection element or pixel of a detector. Under such conditions the number of identifiable signals or pixels with a signal above a certain threshold in a detector correlate substantially to the number of cells being analysed.

The result of the correlation of information is generally presented by displaying a number on an instrument performing the analysis, or on another device connected to the instrument such as a printer or a personal computer.

The assessment result obtained relates to the number of cells of the sample, or to be more precise of the cells to be assessed. Normally the result is the total count of individual cells in the sample. However, the method according to the invention may also be used for counting for example dead or dying cells whereby only cells not viable prior to arranging the sample are counted, and the result relates thereto, independent of any living cells in the sample. Likewise it may be of interest to assess only cells viable prior to arranging the sample, whereby the result only relates thereto.

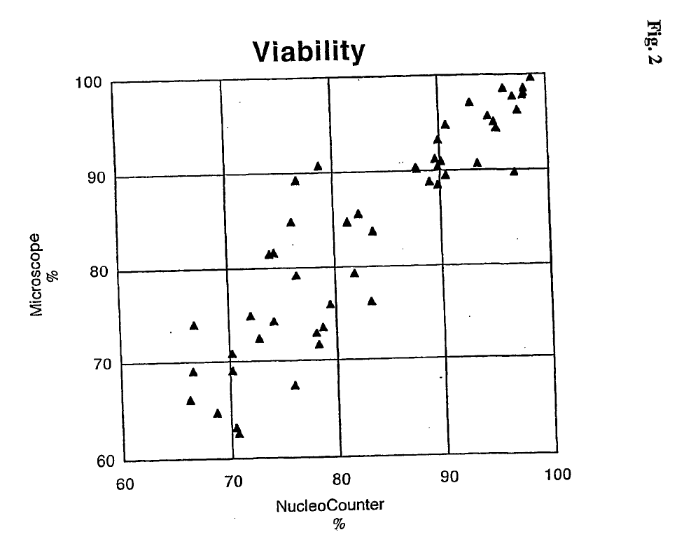

As a living organism cells can undergo various stages. Of particular interest in many embodiments of the present invention are those stages where cells are viable or dead or dying. In particular the ability to assess the number of viable or living cells and/or dead or dying cells or the respective fractions. This can be done in several ways in different embodiments, for instance by determining the total number of cells, viable or dead or dying cells, and then to determine either the number of viable cells and by combining the result, generally to form a fraction of living or viable cells to the total cell count, or a fraction of dead or dying cells to the total cell count. An example of the counting of viable and non-viable cells is disclosed in FIG. 2. The cell samples were analysed before and after treatment with a lysis buffer, giving an estimate of non-viable and total cells, respectively. The manual counting method was based on the Trypan blue exclusion procedure. There is good correlation between the results obtained by the manual method and the results obtained with the present invention.

One method preferred by many embodiments is to add a DNA staining dye to the sample without substantially any further chemical modification of the sample. Dead and dying cells will predominantly be susceptible to this staining, for instance due to ruptured cell membrane or disabled regulating means, while viable cells will to a much less extent be susceptible to staining.

The demise of many species of cells is often in accordance with either the process of necrosis or the process of apoptosis. It is often of interest in the present invention to be able to distinguish between these two processes preferably through the use of a selective methods used to obtain distinguish ability of the cells.

The assessments of one sample may be conducted as a series of assessments, for example the invention includes a method, wherein two volumes of liquid sample are obtained, and a total count of individual cells in one volume is provided and only cells not viable prior to arranging the sample are counted in the other volume. In another combination the invention provides a method, wherein two volumes of liquid sample are obtained, and a total count of individual cells in one volume is provided and only cells viable prior to arranging the sample are counted in the other volume.

The result may also include information, such as a quality parameter, concerning the spatial separation of the identifiable substances, and/or a quality parameter, concerning the reliability of the distinguishability of the identifiable substances.

Magnification

The method and system is particular useful for assessing the number of cells of a sample at a low magnification or enlargement. Thereby it is possible to achieve information relating to a large area of the sample. The magnification may be provided by the focusing means. The magnification of such focusing can be different from 1/1, depending on the set-up of other components of the system, or the particles or sample material used.

Thus, it is often preferred that the spatial representation of the cells exposed onto the array of detection elements is subject to such a linear enlargement that the ratio of the image of a linear dimension on the array of detection elements to the original linear dimension in the exposing domain is smaller than 40:1, normally at the most 20:1, preferably smaller than 10:1 and in many cases even at the most 6:1 or even smaller than 4:1, such as wherein the ratio is in the range between 6:1 and 1:2.

Often embodiments of the invention allow the use of an optical reduction which is 1:1 or less, such as between 1:1 and 1:1.5, or even smaller such as between 1:1 and 1:2, or even as small as 1:4.

By using very low linear enlargement or a one to one exposure the method is rendered more robust with respect to variations in the morphology of the cells to be assessed. Thus by having a low resolution, where electromagnetic signals originating from one cell (i.e. the spatially confined identifiable substances within the cell) are exposed to one, or just a few detection elements, the variation in shape and size of the cells has less importance.

System

The system according to the invention may be any kind of system allowing the signals from the cells to be detected. It is preferred that the system comprises a sample compartment as described above connected to a flow system for flowing the sample from a sample receiving means into the sample compartment. Any pre-treatment of the sample as discussed above may be conducted prior to introducing the sample into the sample receiving means. It is however preferred that some or all of the pre-treatment steps are conducted in one handling, that is in one system or even in one device of the system. In a preferred embodiment the system according to the invention comprises:

a device comprising at least

sample receiving means for receiving said sample

a first preparation chamber

sample flow means for delivering said sample to said first preparation chamber,

at least a first one reagent receiving means for receiving a first reagent,

a first reagent flow means for delivering first reagent from said first reagent receiving means to said first preparation chamber,

a second reagent receiving means for receiving a second reagent,

a second reagent flow means for delivering second reagent from said second reagent receiving means,

a compartment comprising an exposing domain,

a flow channel for delivering said sample and said reagent to said compartment,

a detection device for detecting information relating to each spatially confined identifiable substances,

processing means for processing the information detected,

presentation means for presenting the processed information as a number of cells in the sample.

Systems according to the present invention would normally be systems where the sample can be transported from the exterior and into the instrument and then further within the instrument. Often these systems will include one or more operations such as adding of reagents, mixing, filtration, heating, cooling. The information about the sample is normally recorded in a measurement area, and upon completion the sample is purged, usually to a waste container.

A system can either be a conventional flow system, where the various components of the flow system and the measurement area are a physical part of the instrument or some parts of the flow system, or the entire flow system can be included in a device which only is engaged to the instrument during one or more phases of the analysis.

The present invention is based on the arrangement of at least a part of the sample in such a manner that it extends over a “window” of the device of a substantial area and allows the exposure of signals from the samples. When the spatially resolved electromagnetic radiation signals are to be detected, the device, or at least a part of a window part of the device, is in an engagement with a detection device or an instrument comprising detection means. The engagement is normally the action of placing at least a part of the window in the sensing domain of the detection device, for instance by sliding the device into the sensing domain using one or several guides which at least approximately assure the correct arrangement of the window in the sensing domain. The placing of the device in engagement with the detection device would often be done manually, but in embodiments where the speed of operation and/or the precision of the placing of the device is of importance, the placing of the device could be done automatically, or at least semi automatically, where the placement is performed or controlled by mechanical and/or regulating means.

The flow system according to the invention provides at least one of several operations to be carried out on the samples, said operations being selected from but not limited to transport, mixing with reagent, homogenising of sample and optionally reagent, heat treatment, cooling, sound treatment, ultra sound treatment, light treatment and filtering.

The device may be a disposable device, and in the present context, the term “disposable” indicates that the device in question is adapted to be discarded, or disposed of, after the detection has taken place in the analysis of one sample or a few, often a predetermined number of times. For several embodiments of the invention, it is preferred that the device could be used for several samples, and this could be performed either as one operation or as a series of sequential operations.

The term arranging in relation to means that the device is situated in or engaged with the detection device in a manner whereby the signals from the exposing domain are capable of being exposed to the detection device

In another embodiment the addition is performed by introducing first said liquid sample and afterwards introducing the reaction components or reagents. In this embodiment reaction components or reagents are mostly on liquid form.

In a preferred embodiment of the invention the device contains at least one preparation chamber which allows the mixing of the sample material with a solid or liquid material. In this embodiment the various chemical components to be added to the sample may be added sequentially or simultaneously from their respective reagent receiving means. The addition of components is preferably regulated for example by used of flow means for delivering the components to the preparation chamber.

After the mixing of sample with reagents the mixture is delivered through a flow channel to the sample compartment comprising an exposing domain, said sample compartment being as discussed above.

In order to assure fast assessment of a sample it is of interest to be able to perform analysis shortly after the mixing of any chemical components with sample. This time should therefore be less than 60 seconds, or preferably less than 30 seconds or even as low as 15 seconds and in other preferred situations as low as 10 seconds, and preferably as short as 2 seconds or less and even shorter than 1 second.

In order to flow the sample into or within or out of the disposable device it is preferred to have at least one propelling means provided in the disposable device or in a device with which the disposable device can be engaged. In the latter embodiment it is to be understood that the liquid sample is introduced into the device after engagement with the detection means.

Due to several aspects of any propelling means or the disposable device or the sample analyte material it is preferred that the velocity of the flow into, within, or out of the disposable device is regulated by means of one or more regulating means constituting part of the flow system. Such flow regulating means could be one or more of the selection of stop valves, one way valves, and pressure and/or speed reduction valves.

Preferably the flow regulation means is arranged to function stepwise so that the sample and/or the reagent component may be flowed stepwise through the device. It is furthermore preferred that at least the step of flowing the sample into the exposing domain is carried out in connection with the engagement of the device into the system.

The sample in the device can be flown by the means of a flow system, which can be driven by a pump or a pressurised gas, preferably air, or by causing a pressure difference such that the pressure on the exterior of the inlet is higher than the pressure within at least a part of the device thus forcing the sample to flow through the inlet. In many embodiments of the present invention the flow in said flow system is controlled by one or more valves which can adjust the flow speed of the sample.

When flow only in predominately one direction is preferred, it is of particular interest to use valves which substantially only allow the flow in one direction. Such valves can for instance be placed up- and/or downstream from the sample compartment thus allowing the controlling of the flow condition in the sample compartment. One effect of the use of such valves could be to confine at least a part of the sample in the flow system.

The outlet from the sample compartment can be passed through a flow controlling means, such as a valve, which only allows gas to pass through. One such type of valves which often is preferred, is one which allows gas and air to pass but can close irreversibly when the valve comes in contact with liquid sample. The effect of such valve is to minimise the movement of any sample within the sample compartment during analysis.

Depending on the nature of the sample analyte material and/or the assessment of the cells it is often preferred that the liquid sample is subjected to one or more operations selected from the group consisting of filtration, concentration and magnetic attraction, preferably the disposable device comprising the means for performing such operation or operations.

In another embodiment the information identified relates to the conductivity of a flow of the sample, wherein a change of the conductivity is identified as a cell passing. Thus, the information relating to each individual cell to be assessed may be obtained by measuring conductivity of a flow of the sample through a narrow channel and correlating the change of conductivity to individual cells.

Applications

The method and the system as explained above is characterised by requiring a small sample volume as compared to known methods, which make the invention useful for a wide variety of applications wherein it is of importance that as small a fraction of the cells are withdrawn for tests purposes. Some of the applications are discussed as non-limiting examples below:

Control of processes in a bio-reactor, independent of the cell content of the bio-reactor. In bio-reactors it is often of interest to be informed about the content of total cell count, as well as dead cell count, and living cell count to be informed about the process.

Control of yeast content in bio-reactors or in beer and/or wine-producing processes.

Control of bacteria in bio-reactors and in process water.

In any cell culturing process, wherein the cell count is of interest before, and/or after media shift.

In cell banks the samples received are normally tested with respect to total cell count, as well as dead cell count, and living cell count.

### EXAMPLES

### Example 1

Counting of Mammalian Cells

In order to investigate the applicability of the method of the present invention a number of mammalian cell species were analysed. The result of the estimate of the total cell count obtained by the method of the present invention was compared to the results obtained by reference microscopic analysis using a haemocytometer.

The method according to the present invention that was used was the following: A volume of 200 μl of cell suspension was placed in a vial. A volume of 200 μl of an aqueous reagent containing 2 (w/w) % Triton X-100 and 200 mM citric acid was added to the sample and mixed thoroughly using a vortex mixer. To the solution a volume of 200 μl of an aqueous reagent containing 1% (w/w) Triton X-100 and 250 mM citrate was added and mixed thoroughly using a vortex mixer.

From the final solution a sample of approximately 50 μl was loaded into a NucleoCassette sampling/measuring device (ChemoMetec A/S, Denmark) and the NucleoCassette was then analysed in a NucleoCounter instrument according to the instructions (ChemoMetec A/S, Denmark). The result was reported as cells per volume in the sample/reagent mixture and finally the result was corrected to reflect the estimated total cell count of the cell suspension.

The types of mammalian cells, which were analysed Were the following: 3T3 Swiss Albino, adipocytes, BHK, C13, C134, C146, C199, C214, C69, C867, C916, C927, C928, C946, C970, C975, CHO, CHO-HIR, CHO-K1, COS-7, HEK-293, HT1080, L929, MRC5, NB2-11, NC1-H929, NSO, SP2/0-AG14

The results of the analysis showed that the estimated cell count obtained using a method according to the present invention was in agreement with the reference method, within the limits of 95% significance, estimated in accordance to Poisson statistics.

### Example 2

Estimate of Total Count

In order to investigate the reliability of a method according to the present invention a number of NSO cells (mouse myeloma cells) from a 10 litre bioreactor were analysed. The result of the estimate of the total cell count obtained by a method of the present invention was compared to the results obtained by reference microscopic analysis using a haemocytometer.

The method according to the present invention that was used was the following: A volume of 200 μl of cell suspension from the bioreactor was placed in a vial. A volume of 400 μl of an aqueous reagent containing 2 (w/w) % Triton X-100 and 200 mM citric acid was added to the sample and mixed thoroughly using a vortex mixer. To the solution a volume of 400 μl of an aqueous reagent containing 1% (w/w) Triton X-100 and 250 mM citrate was added and mixed thoroughly using a vortex mixer.

From the final solution a sample of approximately 50 μl was loaded into a NucleoCassette sampling/measuring device (ChemoMetec A/S, Denmark) and the NucleoCassette was then analysed in a NucleoCounter instrument according to the instructions (ChemoMetec A/S, Denmark). The result was reported as cells per volume in the sample/reagent mixture and finally the result was corrected to reflect the estimated total cell count of the cell suspension.

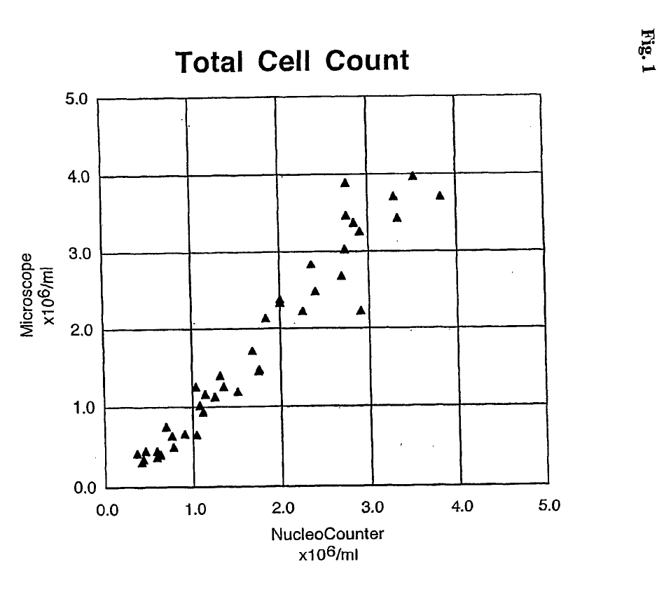

The results of the comparison is given in FIG. 1. The figure shows that there is in general a good agreement between the two methods. The observed correlation coefficient was r=0.96.

In FIG. 2 it is shown that the variation is much less with the method according to the present invention (NucleoCounter) than with the manual method (Microscope). Compare the length of the bars in vertical and horizontal direction.

### Example 3

Sample Pre-treatment

Biological cells, such as virus, bacteria, animal cells, yeast, etc., can have varying properties dependent on the biological species and/or the chemical and for physical environment the biological particles exist in. Dependent on the nature of the sample and biological cells in question various embodiments of the present invention can have different impact on the result with respect to the correctness of the result.

One preferred method according to the present invention is the treatment of a sample of DNA containing cells, such as bacteria, mammalian cells or yeast, is to mix a sample with a lysing reagent, one preferred method consisting of the addition of two reagents, comprised of A) 2% Triton X-100 and 200 mM citric acid and B) 1% Triton X-100 and 250 mM citrate. This method has the effect to make plasma membrane of cells permeable to reagents such as DNA staining dye.

Another property of this method is that cells, which are present in aggregates, such as commonly found with cells, such aggregates become fully or partly separated.

Often the cells themselves becomes fully or partly separated and then the nucleus part of the cell becomes freely suspended. If the nuclei are to be used to enumerate the cells present in a sample then the problem of cellular aggregates is substantially solved.

On the other hand one occasionally comes across a species of cells, which under given conditions form cellular aggregates which are not fully separated after the reagent treatment. One preferred method according to the present invention can further assist in dissolving such cellular aggregates by subjecting the sample to ultrasonic agitation.

The present example illustrates the effect of treating a sample containing cells with ultra-sound with the purpose of dissolving cellular aggregates. Two samples of CHO cells where prepared in T-flasks where one of the samples (I) was cultured to achieve 60% confluence or less, i.e. conditions where cells were predominantly present in one layer, while the other sample (II) was cultured to further to achieve over-confluence, e.i. condition where the cells where present in more than one layer. Sample I is thus expected to be virtually free of cell aggregates after treatment with trypsine and Reagent A and Reagent B while sample 11 is expected to contain cell aggregates even after treatment with Reagent A and Reagent B.

As a source of ultrasonic agitation was used an ultrasonic water bath of the type Branson 1510 (Branson Ultrasonic Corporation, USA). The level of water in the bath was adjusted until the ultrasonic agitation caused vigorous ripples on the surface. Under these conditions the agitation of the ultrasonic water bath is substantially effective. In order to test the efficiency of the agitation a cell suspension in trypsinated media was immersed in the water bath for about 20 seconds. Upon performing an estimate of the total cell count of this sample the result was virtually zero despite that a sample from the same suspension, which had not been subjected to the ultrasonic agitation showed high cell count.

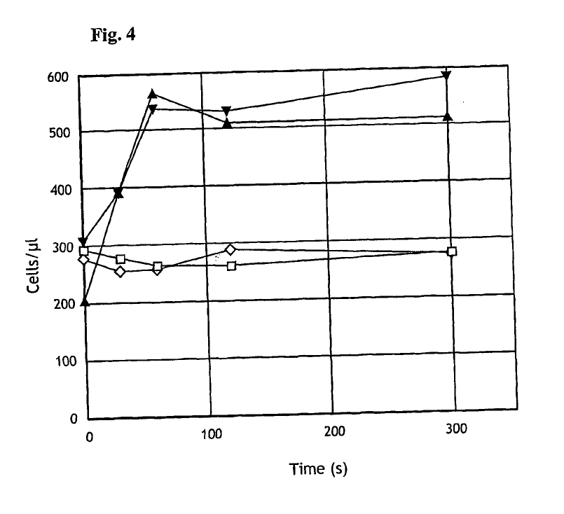

Both samples I and II were subjected to NucleoCounter Reagent A and Reagent B according to instructions for the NucleoCounter (ChemoMetec A/S, Denmark) and the total cell count estimated. Sample II was trypsinated with double portion of trypsine. Then the samples where subjected to ultrasonic agitation for a period of time and the total count estimated again. The result of the estimated total cell count is given in FIG. 4.

FIG. 4 shows that sample I (white squares and diamonds) is virtually unaffected by the ultrasonic treatment, which indicates that at under the conditions used then the cells and/or cell nuclei are stable with respect to ultrasonic agitation. Sample II (black triangles) shows a consistent increase in the cell count from samples not exposed to ultrasonic agitation (T=0 sec.) through the initial phase of the ultrasonic agitation (T<100 sec.). Upon extended ultrasonic treatment Sample It shows a stable cell count indicating that any cell aggregate not separated by the chemical reagents has been separated by ultrasonic agitation.

1. A method for the assessment of the number of cells in a liquid sample, said sample comprising at least one species of cells being selected from a variety of species, cells of each of said species being substantially identical with respect to morphological properties while the inter-species morphological properties may vary, each cell being assessed containing substantially identical spatially confined identifiable substances, such as DNA, comprising the steps of
sampling a volume of the liquid sample of cells,
establishing conditions for making the identifiable substances susceptible to being distinguishable,
establishing conditions providing substantially spatial separation of at least the spatially confined identifiable substances being susceptible for distinguishing, including lysing of the cell membranes of said cells,
optionally staining at least substantially all spatially confined identifiable substances,
identifying information relating to substantially each individual cell to be assessed, and
correlating the information to the number of individual cells in the samples.

2. The method according to claim 1, wherein the variety of species of cells is selected from one of the taxonomic groups: animal cells, yeast cells, fungal cells, plant cells, algae, plasmodia, bacteria, virus.

3. The method according to claim 2, wherein the variety of species of cells are selected from one of the taxonomic groups: animals, including mammal, fish, insect, reptile, preferably wherein the variety of species of cells are mammalian.

4. The method according to any of the preceding claims, wherein the sample is a sample selected from cell cultures, waste water, body fluids such as blood or urine.

5. The method according to any of the preceding claims, wherein the inter-species morphological difference is a variation in size on the order of factor 1.2, preferably more than a factor of 1.2 such as a factor of 1.5, more preferably by a factor of more than 2, more preferably by a factor of more than 4.

6. The method according to any of the preceding claims, wherein the inter-species morphological difference is a variation in shape selected from rod-like, circular, spherical.

7. The method according to any of the preceding claims, wherein the inter-species morphological difference is a variation in symmetry of the cells.

8. The method according to any of the preceding claims, wherein the spatially confined identifiable substance is located in a cell nucleus.

9. The method according to any of the preceding claims, wherein the spatially confined identifiable substance is nucleotides, preferably nucleotides in the cell nucleus.

10. The method according to any of the preceding claims, wherein the condition for making the identifiable substances susceptible to being distinguishable includes making the cells or cell membranes permeable.

11. The method according to any of the preceding claims wherein the condition for making the identifiable substances susceptible to being distinguishable includes partially lysing the cells.

12. The method according to any of the preceding claims, wherein the condition for making the identifiable substance susceptible to being distinguishable includes selective staining of one or several receptors on the surface of the cells or in the interior of the cells.

13. The method according to any of the preceding claims, wherein the conditions providing substantially spatial separation of at least the spatially confined identifiable substances being susceptible for distinguishing includes separation of cell aggregates into cells or nuclei.

14. The method according to any of the preceding claims, wherein the lysing conditions are selected to obtain lysing of cell membranes, but not lysing of nucleus of the cell, preferably where the lysing of the cell membranes is to an extent where the nucleus of the cell is separated from the remaining components of the cell.

15. The method according to any of the preceding claims, wherein the lysis step includes adding a reagent to obtain a low pH of the final solution.

16. The method according to any of the preceding claims, wherein the lysis step includes adding a reagent to obtain a pH of the final solution of between 2 and 6.

17. The method according to claim 15 or 16, wherein the pH of the final solution is further adjusted prior to analysis.

18. The method according to any of the preceding claims, wherein the conditions providing substantially spatial separation of the spatially confined identifiable substances being susceptible of being distinguished included an ultrasound treatment.

19. The method according to claim 18, wherein the ultrasound treatment is performed after adding a reagent to the cells causing the nucleus not to disintegrate upon ultrasound treatment, preferably wherein said reagent has the effect of adjusting the pH of the sample, more preferably wherein said reagent comprises an acid, such as a polyvalent acid, preferably citric acid.

20. The method according to claim 18, wherein the duration of the ultrasound treatment is from 20 seconds to 5 minutes, more preferably from 30 seconds to 2 minutes.

21. The method according to claim 18, wherein the ultrasound treatment provides an effect of 50 to 120% of the effect required to destroy the cells under similar circumstances.

22. The method according to any of the preceding claims, wherein a reagent added to the cell sample prior to identifying information relating to substantially each individual cell to be assessed contains t-Octylphenoxy polyethoxyethanol (Triton® X-100), preferably where the t-Octylphenoxy polyethoxyethanol concentration of the final solution is between 0.05% and 5%, more preferably where the t-Octylphenoxy polyethoxyethanol concentration of the final solution is between 0.1% and 2%, more preferably where the t-Octylphenoxy polyethoxyethanol concentration of the final solution is between 0.2% and 1.5%, more preferably where the t-Octylphenoxy polyethoxyethanol concentration in the final solution is between 0.75 and 1.25%, such as 1%.

23. The method according to any of the preceding claims, wherein a reagent added to time cell sample prior to identifying information relating to substantially each individual cell to be assessed contains CPC (Cetyl Pyridinium Chloride) preferably where the CPC concentration of the final solution is between 0.05% and 5%, more preferably where the CPC concentraton of the final solution is between 0.1% and 2%, more preferably where the CPC concentration of the final solution is between 0.2% and 1%.

24. The method according to any of the preceding claims, wherein a reagent added to the cell sample prior to identifying information relating to substantially each individual cell to be assessed contains a protease enzyme, preferably Typsin, more preferably where the Trypsin concentration of the final solution is between 0.05% and 5%, more preferably where the Trypsin concentration of the final solution is between 0.1% and 2%, more preferably where the Trypsin concentration of the final solution is between 0.2% and 1%.

25. The method according to any of the preceding claims, wherein a reagent added to the cell sample prior to identifying information relating to substantially each individual cell to be assessed contains a cell-wall degrading reagent.

26. The method according to claim 25, wherein said cell-wall degrading reagent comprises at least one enzyme, such as lysozyme, cellulase, pectinase, hemicellulase where the enzyme concentration of the final solution is between 0.05% and 5%, more preferably where the enzyme concentration of the final solution is between 0.1% and 2%, more preferably where the enzyme concentration of the final solution is between 0.2% and 1%.

27. The method according to claim 25, wherein said cell-wall degrading reagent comprises a strong acid capable of hydmlysing the cell wall optionally at elevated temperature.

28. The method according to any of the preceding claims, wherein a reagent added to the cell sample prior to identifying information relating to substantially each individual cell to be assessed containing Citric acid, preferably where the Citric acid concentration of the final solution is between 1 mM and 1000 mM, more preferably where the Citric acid concentration of the final solution is between 10 mM and 500 mM, more preferably where the Citric acid concentration of the final solution is between 50 mM and 250 mM, such as between 100 and 200 mM, for example approximately 150 mM.

29. The method according to any of the preceding claims, wherein a reagents added to the cell sample prior to identifying information relating to substantially each individual cell to be assessed has the effect of binding ions, preferably where the reagent contains one or several of the following: Citric acid, EDTA.

30. The method according to any of the preceding claims, wherein a reagent added to the cell sample prior to identifying information relating to substantially each individual cell to be assessed has the effect of adjusting the pH of the solution, preferably where the pH of the final solution is between 1 and 7, more preferably where the pH of the final solution is between 2 and 6, more preferably where the pH of the final solution is between 3 and 5.

31. The method according to any of the preceding claims, wherein a reagent added to the cell sample prior to identifying information relating to substantially each individual cell to be assessed has the effect of adjusting the pH of the solution, where the pH of the final solution is between 3 and 9, more preferably where the pH of the final solution is between 4 and 8, more preferably where the pH of the final solution is between 5 and 7.

32. The method according to any of the previous claim, wherein adjusting of pH is achieved by the addition of pH buffer, preferably where the pH buffer is selected from one or more of the following: Citrate buffer, Phosphate buffer, HEPES buffer.

33. The method according to any of the preceding claims, wherein the cells are stained before detection.

34. The method according to claim 33, wherein the staining is selected from molecules giving rise to one or several of the following phenomena: attenuation of electromagnetic radiation, photoluminescence when illuminated with electromagnetic radiation, scatter of electromagnetic radiation, raman scatter.

35. The method according to claim 33, wherein the staining is selected from one or more nucleic acid dyes and/or one or more potentiometric membrane dyes is added.

36. The method according to claim 33, wherein the staining is selected from acridine orange (CAS-65-61-2/CAS-10127-02-3), cyanine dyes (e.g. TOTO™-1 iodide CAS#: 143 413-84-7-Molecular Probes, YO-PRO™-1 iodide CAS#: 152 068-09-2-Molecular Probes), indoles and imidazoles (e.g. Hoechst 33258 CAS#: 023 491-454, Hoechst 33342 CAS#: 023 491-52-3, DAPI CAS#: 28718-90-3, DIPI (4′,6-(diimidazolin-2-yl)-2-phenylindole), in particular propidium iodide CAS#: 25535-16-4).

37. The method according to any of the preceding claims, wherein the analysis is performed shortly after the mixing of any chemical components with the sample, preferably less than 60 seconds less than 30 seconds, such as 15 seconds, 10 seconds, 2 seconds or less than 1 second after the mixing of any chemical components with the sample.

38. The method according to any of the preceding claims, wherein the information relating to each individual cell to be assessed is obtained by performing one or more exposures of spatially resolved electromagnetic signals from the sample onto an array of active detection elements.

39. The method according to any of the preceding claims, wherein the information relating to cells is electromagnetic signals.

40. The method according to any of the preceding claims, wherein the information relating to cells is fluorescence signals.

41. The method according to any of the preceding claims, wherein the sample is arranged in a sample compartment.

42. The method according to claim 41, wherein the sample compartment has a wall part defining an exposing area, the wall part allowing electromagnetic signals from the sample in the compartment to pass through the wall to the exterior.

43. The method according to claim 41, wherein the interior of the sample compartment has an average thickness of between 20 μm and 200 μm.

44. The method according to claim 41, wherein the sample compartment has dimensions, in a direction substantially parallel to the array of detection elements, in the range between 1 mm by 1 mm and 10 mm by 10 mm.

45. The method according to claim 41 wherein the volume of the liquid sample from which electromagnetic radiation is detected on the array is in the range between 0.01 μl and 20 μl.

46. The method according to claim 41, wherein the sample in the sample compartment is at stand still during the exposure.

47. The method according to claim 38, wherein the array of detection elements is arranged in such a way that a series of detection elements form a substantially straight line.

48. The method according to claim 38, wherein the array of detection elements is arranged in two directions in such a way that the detection elements form a series of substantially parallel straight lines, the series forming a rectangle.

49. The method according to claim 38, wherein the exposure of spatially resolved electromagnetic signals onto the array of detection elements is preformed by focusing spatially resolved electromagnetic signals from at least a part of the exposing domain onto the array of detection elements by means of a focusing means.

50. The method according to any of the preceding claims wherein electromagnetic radiation from the individual particles the parameter or parameters of which is/are to be assessed are exposed on at the most 1000 detection elements.

51. The method according to claim 38, wherein the spatial representation of the cells exposed onto the array of detection elements is subject to such a linear enlargement that the ratio of the image of a linear dimension on the array of detection elements to the original linear dimension in the exposing domain is smaller than 40:1, normally at the most 20:1, preferably smaller than 10:1 such as at the most 6:1 or smaller than 4:1 such as wherein the ratio is in the range between 6:1 and 1:2.

52. The method according to claim 38, comprising the use of an optical reduction which is less than 1:1, such as between 1:1 and 1:1.5, or even smaller such as between 1:1 and 1:2, or 1:4.

53. The method according to any of the preceding claims, wherein the correlation to the number of cells is conducted without input of any apriori information about the species of cells within a given taxonomic group being analysed.

54. The method according to any of the preceding claims, wherein the correlation to the number of cells is conducted without use of any morphological information about the species of cells within a given taxonomic group derivable from the detection.

55. The method according to any of the preceding claims, wherein a total count of individual cells in the sample is provided.

56. The method according to any of the preceding claims, wherein a quality parameter, concerning the spatial separation of the identifiable substances is provided.

57. The method according to any of the preceding claims, wherein a quality parameter, concerning the reliability of the distinguish ability of the identifiable substances is provided.

58. The method according to any of the preceding claims 1-54, wherein only cells not viable prior to sampling the sample are counted.

59. The method according to any of the preceding claims 1-54, Wherein only cells viable prior to sampling are counted.

60. The method according to any of the preceding claims 1-54, wherein only cells undergoing apoptotic process prior to sampling are counted.

61. The method according to any of the preceding claims 1-54, wherein only apoptotic bodies are counted.

62. The method according to any of the preceding claims 1-54, wherein only cells undergoing necrosis process prior to sampling are counted.

63. The method according to any of the preceding claims, wherein two volumes of liquid sample are obtained, and a total count of individual cells in one volume is provided and only cells not viable prior to sampling the sample are counted in the other volume.

64. The method according to any of the preceding claims, wherein two volumes of liquid sample are obtained, and a total count of individual cells in one volume is provided and only cells viable prior to sampling the sample are counted in the other volume.

65. The method according to any of the preceding claims, wherein two volumes of liquid sample are obtained, and a total count of cells in one volume is provided and only cells not viable prior to sampling the sample are counted in the other volume.

66. A system for the assessment of the number of cells in a liquid sample, said sample comprising at least one species of cells being selected from a variety of species, cells of each of said species being substantially identical with respect to morphological properties, while the inter-species morphological properties may vary, each cell being assessed containing substantially identical spatially confined identifiable substances, comprising:
a device comprising at least
sample receiving means for receiving said sample
a first preparation chamber
sample flow means for delivering said sample to said first preparation chamber,
at least a first one reagent receiving means for receiving a first reagent,
a first reagent flow means for delivering first reagent from said first reagent receiving means to said first preparation chamber,
a second reagent receiving means for receiving a second reagent,
a second reagent flow means for delivering second reagent from said second reagent receiving means,
a compartment comprising an exposing domain,
a flow channel for delivering said sample and said reagent to said compartment,
a detection device for detecting information relating to each spatially confined identifiable substances,
processing means for processing the information detected,
presentation means for presenting the processed information as a number of cells in the sample.

67. The system according to claim 66, further comprising means for subjecting a sample to ultrasound.

68. The system according to claim 67, wherein the means for subjecting a sample to ultrasound is positioned in connection with a flow system.
